(19) United States
(12) Patent Application Publication
(10) Pub. No.: US 2004/0132039 A1
(43) Pub. Date: Jul. 8, 2004

(54) Screening method comprising combination of discrete dimension assay testing of actives, gene mutation and application assay testing of selected actives

(76) Inventors: Soren Kjaerulf, Vanlose (DK); Michael Lamsa, Davis, CA (US); Thomas Thisted, Rungsted Kyst (DK); Peter Kamp, Hansen Lejre (DK); Henrik Pedersen, Bagsvaerd (DK); Henrik Frisner, Copenhagen (DK)

Correspondence Address: NOVOZYMES NORTH AMERICA, INC., 500 FIFTH AVENUE, SUITE 1600, NEW YORK, NY, 10110, US

(21) Appl. No.: 10/471,269

(22) PCT Filed: Mar. 12, 2002
(86) PCT No.: PCT/DK02/00160

(51) Int. Cl. C12Q 1/68

(52) U.S. Cl. 435/6

(57) ABSTRACT

Screening method comprising combination of discrete dimension assay testing of actives, gene mutation and application assay testing of selected actives. The present invention relates to a screening method for identifying actives with improved performance comprising: (1) screening a library of actives encoded by nucleotide sequences in at least two different discrete dimension assays employing selected parameters and selecting the actives exhibiting improved properties in the discrete dimension assays, (2) collecting nucleotide sequences encoding the selected actives, (3) mutating collected nucleotide sequences, (4) screening actives encoded by the mutated nucleotide sequences in an application assay and selecting actives, which exhibit improved performance.

### FIELD OF THE INVENTION

The present invention relates to an improved method for screening for modified actives or nucleotide sequences encoding modified actives, wherein the modified actives have improved performance in application.

### BACKGROUND OF THE INVENTION

Many methods for screening for actives within biotechnology have been reported. In the pursuit of improved actives, screening methods of the prior art have usually been carried out by either testing the performance of actives in real like application assays (application assays) or by testing actives in more or less artificial screening methods involving variation in one or more selected parameters (discrete dimension assays). When using application assays the practitioner must test a huge number of potential candidates in assays, which are usually expensive, slow and laborious. Using discrete dimension assays involving variation of one or more selected parameters may be less costly and fast, but the practitioner will experience that many of the candidate actives, which show promising results in these assays, will not show the desired improvement in the real application because the function of the active in the real application is much more complicated than the function in the artificial test assay.

The function of actives in application is usually affected by a complex collection of known and unknown conditions or parameters of the application. The effect of these conditions on the active may be either positive or negative on the performance of the active in that application.

When screening for new actives having improved performance in an application among a huge amount of candidate actives it is important and useful in the screening process to mimic the conditions or parameters of the real application to ensure that the performance of the new actives found is indeed improved in the real application.

However, conducting screening at real application conditions is costly and time consuming, while screening made with fast and cheap artificial assays will yield unreliable results.

### SUMMARY OF THE INVENTION

One object of the present invention is to provide a screening method, which limit the number of potential candidates to be tested in assays mimicking the intended application of the active. In modern screening within biotechnology the amount of candidate actives and genes encoding them is enormous—especially in view of modern methods for recombining or mutating candidate actives and genes encoding them. By limiting the number of candidate actives to be tested the speed of screening process and the cost involved may be improved considerably.

Another object of the invention is to provide a screening method wherein the reliability of performance of found actives is improved.

The present invention provides in a first aspect a screening method for identifying actives with improved performance comprising:

(1) screening a library of actives encoded by nucleotide sequences in at least two different discrete dimension assays employing selected parameters and selecting the limited number of actives exhibiting improved properties in the discrete dimension assays,

(2) collecting nucleotide sequences encoding the selected actives,

(3) mutating collected nucleotide sequences,

(4) screening actives encoded by the mutated nucleotide sequences in an application assay and selecting actives, which exhibit improved performance.

In a second aspect the invention relates to an active obtained by the said screening method.

### DETAILED DESCRIPTION OF THE INVENTION

### Definitions

The term “discrete dimension assay” as used herein is to be understood as an assay, wherein the effect of a discrete number of parameters on an active is tested. It is further to be contemplated that the term parameter is to be understood as one or more selected conditions under which the performance of the active is intended to be investigated while all other conditions are selected as being suitable for the function of the active.

In a particular embodiment the parameters are selected from an application assay for testing the performance of an active. If for example the application of the-active is a washing process and the active is an enzyme the testing of the effect of the presence of e.g. a surfactant on the activity of the enzyme under conditions otherwise suitable for the activity of the enzyme would constitute a discrete dimension assay for that active and that application. Another example would be to test the effect of high alkalinity as employed in washing applications on the enzyme activity under conditions otherwise suitable for the activity of the enzyme.

In particular embodiment the number of parameters selected for the discrete dimension assays, wherein the term discrete is to be understood as a number less than the number of parameters in an application, is between 0 and 10, such as between 0 and 5, especially 1, 2, 3 or 4.

In a particular embodiment only one parameter is selected for the discrete dimension assay and thus only the effect of one parameter on the active is tested in the discrete dimension assay. Selecting only one parameter offers the special advantage that those actives having the most improved performance in respect of the chosen single parameter may be identified and selected. In contrast thereto employing a discrete dimension assay involving two or more parameters will result in the finding of actives having improved performance with respect to two or more parameters in combination. Thus for example an active may have a high performance improvement with respect to one parameter, but a performance decrease with respect to one or more other parameters. Such an active may not have an overall performance improvement in respect of the two or more parameters in combination and will therefore typically be deselected as a starting point for making further improvements. However, with the method of the present invention, such active may be found useful as a starting compound for developing actives with improved performance in the real application, e.g. in the application assay. Hence, when identifying improved actives by employing single dimension assays involving two or more parameters those actives will typically have less improved performance for each individual parameter than actives identifying by involving only one parameter.

The term “different discrete dimension assays” refers to more than one discrete dimension assays, wherein a parameter tested in one assay is different from a parameter tested in another, e.g. parameters could be the effect the presence of a surfactant has on the activity of an enzyme, the effect an increase in temperature has on the activity/function of a protein (thermal stability) etc.

The term “application assay” as used herein is to be understood as an assay, wherein the effect from all known and unknown parameters of an application on the performance of an active is tested. If for example the application is a washing process and the active is an enzyme the testing of the performance of the enzyme in a washing machine would constitute an application assay for that active and that application.

The term “improved performance” or “improved properties” is in the context of the present invention to be understood as the ability of an active to perform better or to have properties which enables it to perform better in a discrete dimension assay, an application assay or an application than a given control. For example if a library represents mutations of a parent protein the ability of an active from said library to degrade a substrate faster than the parent protein then said active has an improved performance.

The term “limited number” is in the context of the present invention to be understood as less than the number of actives represented by the library.

### Actives

The actives of the present invention are proteins or peptides, which are encoded by nucleotide sequences and may be produced by expression of the nucleotide sequences in an expression system. The actives of the invention include among others bio-catalysts such as enzymes, therapeutic agents such as hormones, insulin, growth factors, coagulation factors, antibodies, receptors or antibiotics and biocides such as herbicides, pesticides and fungicides.

In a particular embodiment the active is an enzyme. The enzyme classification employed in the present specification with claims is in accordance with Recommendations (1992) of the Nomenclature Committee of the International Union of Biochemistry and Molecular Biology, Academic Press, Inc., 1992.

Accordingly the types of enzymes which may appropriately be screened for include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Examples of oxidoreductases in the context of the invention are peroxidases (EC 1.11.1), laccases (EC 1.10.3.2) and glucose oxidases (EC 1.1.3.4)], while particular transferases are transferases in any of the following sub-classes:

a) Transferases transferring one-carbon groups (EC 2.1);

b) transferases transferring aldehyde or ketone residues (EC 2.2); acyltransferases (EC 2.3);

c) glycosyltransferases (EC 2.4);

d) transferases transferring alkyl or aryl groups, other that methyl groups (EC 2.5); and

e) transferases transferring nitrogenous groups (EC 2.6).

A most particular type of transferase in the context of the invention is a transglutaminase (protein-glutamine gammaglutamyltransferase; EC 2.3.2.13).

Examples of hydrolases in the context of the invention are: Carboxylic ester hydrolases (EC 3.1.1.-) such as lipases (EC 3.1.1.3); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as alpha-amylases (EC 3.2.1.1); peptidases (EC 3.4, also known as proteases); and other carbonyl hydrolases].

In the present context, the term “carbohydrase” is used to denote not only enzymes capable of breaking down carbohydrate chains (e.g. starches) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses): alpha-amylases (3.2.1.1), beta-amylases (3.2.1.2), glucan 1,4-alpha-glucosidases (3.2.1.3), cellulases (3.2.1.4), endo-1,3(4)-beta-glucanases (3.2.1.6), endo-1,4-beta-xylanases (3.2.1.8), dextranases (3.2.1.11), chitinases (3.2.1.14), polygalacturonases (3.2.1.15), lysozymes (3.2.1.17), beta-glucosidases (3.2.1.21), alpha-galactosidases (3.2.1.22), beta-galactosidases (3.2.1.23), amylo-1,6-glucosidases (3.2.1.33), xylan 1,4-beta-xylosidases (3.2.1.37), glucan endo-1,3-beta-D-glucosidases (3.2.1.39), alpha-dextrin endo-1,6-alpha-glucosidases (3.2.1.41), sucrose alpha-glucosidases (3.2.1.48), glucan endo-1,3-alpha-glucosidases (3.2.1.59), glucan 1,4-beta-glucosidases (3.2.1.74), glucan endo-1,6-beta-glucosidases (3.2.1.75), arabinan endo-1,5-alpha-L-arabinosidases (3.2.1.99), lactases (3.2.1.108), chitosanases (3.2.1.132) and xylose isomerases (5.3.1.5).

### The Library

The library may be a library representing any collection of actives. For example the library may be a library representing the actives expressed by a given cell type/organism/species, or it may be a library representing the extracellular, intracellular or cell-surface actives expressed by a given cell type/organism/species, or it may be a library representing an active with a given function expressed by different cell types/organisms/species, or it may be a library representing mutated variants of an active wherein said mutated variants may be generated by any known method within the art of mutating nucleotides.

For example if the active is a protease and it is to be optimized for improved wash performance libraries which may be screened include:

a) a library representing mutations of residues around Ca2+ binding site

b) a library representing mutations changing charged surface residues

c) a library representing mutations located in a active site or involving amino acids located adjacent to amino acids in the active site (so-called second-shell mutations)

d) a library representing mutations in positions with known or presumed effect on thermostability

e) a library representing mutations in positions with known or presumed effect as to promoting autoproteolysis

f) a library representing mutations in positions with known or presumed effect on low temperature enzyme activity identified experimentally or based on homology and sequence alignment with the family of psychrophilic proteases

g) a library of mutations in positions within or adjacent to known or presumed epitopes (regions of antibody interaction and potentially conferring allergenicity or altered immunogenicity (e.g. increased immunogenicity) to the protein of interest) identified experimentally or based on computational epitope mapping such as methods described in e.g. WO 92/10755, WO 00/26230, WO 01/83559 or WO 01/31989

In one embodiment of the present invention the library/libraries may be designed so that they are optimized towards testing of a specific parameter in the discrete dimension assay. For example the above mentioned libraries may typically be used to screen for/in:

a) activity/stability in the presence of chelators (e.g. EGTA)

b) stability in the presence of LAS

c) increased specific activity or altered substrate specificity

d) a thermostability assay

e) a stability assay conducted in a suitable buffer in which autoproteolysis normally would occur

f) enzyme activity at low temperature

g) decreased antibody binding/allergenicity.

Library Preparation

The actives of the present invention being proteins or peptides are encoded by nucleotide sequences (herein after denoted “genes”) and may be produced by expression of the nucleotide sequences in an expression system. In the pursuit for improved actives it is preferred to prepare a library of nucleotide sequences (herein after denoted “gene library”) encoding the actives to be screened and to prepare a corresponding library of actives by expressing each member of the gene library in an expression system. In the context of the invention, the term expression system is to be understood as a system enabling transcription of a gene and translation into the synthesis of the corresponding active. The expression system may be a cell or an in vitro system.

Expression Systems

The expression system may be cellular or an in vitro system. A description of in vitro coupled transcription and translation may be found in Ohuchi, S. et al.; In vitro method for generation of protein libraries using PCR amplification of a single DNA molecule and coupled transcription/translation; Nucleic Acid research, 1998, vol. 26. No. 19, pp. 4339-4346 or Ellman et al., Methods in Enzymol.1991; vol. 202; pp. 301-337. In the case of a cellular expression system, the cell may be e.g. a wild type cell or it may be a cell from a population of transformed host cells or clones thereof comprising a gene library prepared according to methods known to the art (e.g. described vide infra).

The Host Cell

In the context of the invention, the term “host cell” is to be understood as a cell, which may host and may express an inserted gene from a gene library. The host cell may be any cell capable of hosting and expressing a gene from a gene library.

In particular a host cell does not in itself contain or express genes encoding the active. This cell characteristic may either be a natural feature of the cell or it may be obtained by deletion of such genes as described e.g. in Christiansen et al., Xanthine metabolism in Bacillus subtilis: Characterization of the xpt-pbuX operon and evidence for purine and nitrogen controlled expression of genes involved in xanthine salvage and catabolism, Journal of Bacteriology, 179(8), pp 2540-2550, 1997 or Stoss et al., Integrative vector for constructing single copy translational fusions between regulatory regions of Bacillus subtilis and the bgaB reporter gene encoding a heat stable beta-galactosidase, FEMS Microbiology Letters, 150(1), pp 49-54, 1997. In particular host cells may be bacterial cells, such as strains of E. coli and Bacillus (e.g. Bacillus subtilis or Bacillus sp.) or eukaryotic cells such fungal cells, e.g. from a strain of Aspergillus or yeasts (e.g. S. cerevisae or Picia pastoris).

Preparation of the Nucleotide Sequence Library

Preparation of a gene library can be achieved by use of known methods.

Procedures for extracting genes from a cellular nucleotide source and preparing a gene library are described in e.g. Pitcher et al., “Rapid extraction of bacterial genomic DNA with guanidium thiocyanate”, Lett. Appl. Microbiol., 8, pp 151-156, 1989, Dretzen, G. et al., “A reliable method for the recovery of DNA fragments from agarose and acrylamide gels”, Anal. Biochem., 112, pp 295-298, 1981, WO 94/19454 and Diderichsen et al., “Cloning of aldB, which encodes alpha-acetolactate decarboxylase, an exoenzyme from Bacillus brevis”, J. Bacteriol., 172, pp 4315-4321, 1990.

Procedures for preparing a gene library from an in vitro made synthetic nucleotide source can be found in (e.g. described by Stemmer, Proc. Natl. Acad. Sci. USA, 91, pp. 10747-10751, 1994 or WO 95/17413).

Insertion of a Gene Library into the Host Cell

Procedures for transformation of a host cell by insertion of a plasmid comprising a gene from a gene library is well known to the art, e.g. Sambrook et al., “Molecular cloning: A laboratory manual”, Cold Spring Harbor lab., Cold Spring Harbor, N.Y., 1989, Ausubel et al.(eds.), Current protocols in Molecular Biology, John Wiley and Sons, 1995 and Harwood and Cutting (eds. ), “Molecular Biological Methods for Bacillus”, John Wiley and Sons, 1990.

In a particular embodiment of the invention the plasmid to be inserted into a host cell also contains a gene (denoted as an antibiotic marker), which may enable resistance of, a transformant to an antibacterial or antifungal agent e.g. an antibiotic. Resistance to chloramphenicol, tetracycline, kanamycin, ampicillin, erythromycin or zeocin is preferred.

Manipulating the Nucleotide Sequences of a Library

In a particular embodiment the genes of a gene library may before, during or after initiating the screening be subjected to alterations and or mutations. Generation of libraries of genes encoding variants of actives can be done in a variety of ways:

(1) Error prone PCR employs a low fidelity replication step to introduce random point mutations at each round of amplification (Caldwell and Joyce (1992), PCR Methods and Applications vol.2 (1), pp.28-33). Error-prone PCR mutagenesis is performed using a plasmid encoding the wild-type, i.e. wt, gene of interest as template to amplify this gene with flanking primers under PCR conditions where increased error rates leads to introduction of random point mutations. The PCR conditions utilized are typically: 10 mM Tris-HCl, pH 8.3, 50 mM KCl, 4 mM MgCl₂, 0.3 mM MnCl₂, 0.1 mM dGTP/dATP, 0.5 mM dTTP/dCTP, and 2.5 u Taq polymerase per 100 μl of reaction. The resultant PCR fragment is purified on a gel and cloned using standard molecular biology techniques.

(2) Oligonucleotide directed mutagenesis in single codon position (including deletions or insertions), e.g. by SOE-PCR is described by Kirchhoff and Desrosiers, PCR Methods and Applications, 1993, 2, 301-304. This method is performed as follows: Two independent PCR reactions are performed with 2 internal, overlapping primers, wherein one or both contain a mutant sequence and 2 external primers, which may encode restriction sites, thereby creating 2 overlapping PCR fragments. These PCR fragments are purified, diluted, and mixed in molar ratio 1:1. The full length PCR product is subsequently obtained by PCR amplification with the external primers. The PCR fragment is purified on gel and cloned using standard molecular biology techniques.

(3) oligonucleotide directed randomization in single codon position, such as saturation mutagenesis, may be done e.g. by SOE-PCR as described above, but using primers with randomized nucleotides. For example NN(G/T), wherein N is any of the 4 bases G,A,T or C, will yield a mixture of codons encoding all possible amino acids.

(4) Combinatorial site-directed mutagenesis libraries may be employed, where several codons can be mutated at once using (2) and (3) above. For multiple sites, several overlapping PCR fragments are assembled simultaneously in a SOE-PCR setup.

(5) Another protocol employs synthetic gene libraries preparation. Wild type, i.e. wt, genes can be assembled from multiple overlapping oligonucleotides (typically 40-100 nucleotides in length; (Stemmer et al., (1995), Gene 164, 49-53). By including mixtures of wt and mutant variants of the same oligo at various positions in the gene, the resulting assembled gene will contain mutations at various positions with mutagenic rates corresponding to the ratios of wt to mutant primers.

(6) Still another method employs multiple mutagenic primers to generate libraries with multiple mutated positions. First an uracil-containing nucleotide template encoding a polypeptide of interest is generated and 2-50 mutagenic primers corresponding to at least one region of identity in the nucleotide template are synthezised so that each mutagenic primer comprises at least one substitution of the template sequence (or: insertion/deletion of bases) resulting in at least one amino acid substitution (or insertion/deletion) of the amino acid sequence encoded by the uracil-containing nucleotide template. The mutagenic primers are then contacted with the uracil-containing nucleotide template under conditions wherein a mutagenic primer anneals to the template sequence. This is followed by extension of the primer(s) catalyzed by a polymerase to generate a mixture of mutagenized polynucleotides and uracil-containing templates. Finally, a host cell is transformed with the polynucleotide and template mixture wherein the template is degraded and the mutagenized polynucleotide replicated, generating a library of polynucleotide variants of the gene of interest.

(7) Libraries may be created by shuffling e.g. by recombination of two or more wt genes or genes encoding variant proteins created by any combination of methods (1)-(6) (above) by DNA shuffling.

Picking of the Active

If the active is an enzyme it may be picked from the library by utilizing an agar plate method where the media contains a substrate that acts as a detection system for the enzyme being expressed. Typically, a clearing zone or activity zone is produced around the colony, distinguishing the colony growing in that zone from colonies that do not produce an active enzyme. The choice of substrate depends on the type of enzyme, i.e. it should be a substrate for the particular enzyme. For example if the enzyme is a protease skim milk may be used as a substrate or if it is an amylase starch may be used as a substrate.

The clearing zone of actives can be enhanced and localized by inoculating the organism into a small hole punched into the surface of the agar. This hole acts as a containment device for the organism, minimizing growth, while producing a highly visible clearing zone that is more easily detected by the colony-picking robot. Furthermore, due to the discrete localization of the growth, more colonies may be screened on a single plate. This method can be applied to any analytical method producing a clearing zone.

In particular, the proportion between the number of cells/organisms and the number of holes on the plate may be optimized to increase the probability of only one cell/organism per hole being the ancestor of a colony. In particular the organisms may be inoculated onto a plate containing 10-fold more holes than the total number of cells/organisms applied to the plate. Using Poisson statistics it can be shown that greater than 9 in 10 colonies which grow will have arisen from a single cell when the proportion between the total number of cells/organisms and the number of holes is 1:10.

Another way of picking actives from a library is to add a particulate substrate, i.e. a substrate bound/coupled to a compound which can easily be detected, e.g. a dye bound to an insoluble substrate, to an agar plate into which holes have been punched, such that all of the substrate is forced into the holes on the plate. The host organism may be applied at the same time as the substrate. Cleavage of the substrate will release the tag into the agar and thereby generate a visible zone around the host organism. Host organisms expressing the active of interest (capable of hydrolyzing the substrate) can then be distinguished from organisms which do not express said active by the presence of a visible zone around the former host organism. Also for this method the proportion between the number of cells/organisms and the number of holes on the plate may be optimized to increase the probability of only one cell/organism per hole being the progenitor of a colony using the Poisson statistical method. In particular, the organisms may be inoculated onto a plate containing 10-fold more holes than the total number of cells/organisms applied to the plate.

### The Screening Method

Discrete Dimension Assays

The first screening step in the present invention is to screen a prepared gene library expressing actives in at least two different discrete dimension assays to identify actives having improved properties with respect to each of the particular parameters of these assays. Potential candidates of actives, i.e. actives showing improved properties in a discrete dimension assay, need not to show improved properties in both discrete dimension assays. Improved properties in only one of the assays suffice for an active to be selected as a candidate for the next screening steps.

In one embodiment of the present invention the first screening step comprises screening of different libraries, e.g. between 2 to 5 different libraries, in different discrete dimension assays. In particular the different libraries may be optimized for different specific condition, e.g. the presence of a chelator, thermostability etc. other examples are given above.

For example if a protease is to be optimized for improved performance in washing one library representing mutations around the Ca2+ site of the protease may be screened in an assay wherein the presence of a chelator, such as EGTA, is tested on the protease stability/activity and another library, e.g. representing mutations changing charged surface residues of a protease may be screened in an assay wherein the presence of LAS is tested on the stability of the protease. Subsequently, actives with improved properties in the assays are collected and recombined to create a new library which may be screened in a discrete dimension assay or in an application assay. By recombining the actives selected for improved performance in the presence of different parameters a library representing actives which have improved performance in the presence of more than one parameter can be generated. For example in the above case a library representing a protease with improved performance in the presence of a chelator and LAS.

Relevant discrete dimension assays should in a particular embodiment be chosen with respect to the final performance in relevant application assays. In particular parameters of the discrete dimension assays are those known to be included in the application of the active, i.e. those, which are also part of the complex application assay to be performed later in the method of the present invention. Accordingly the design and choice of parameters in discrete dimension assays in the search for a particular type of active is in a further particular embodiment chosen by “breaking down” the complex application assay for that type of active, having multiple parameters, into a number of discrete dimension assays having only a single parameter. For example, if screening is intended for new enzyme actives used in laundry detergents, examples of relevant discrete dimension assays could be:

(1) Assays testing the performance of enzymes with respect to their specific activity at washing relevant conditions. Parameter(s) may be selected from temperature, pH, low Ca2+ levels etc. or a combination thereof. Ness et al. (1999) Nature Biotechnology 17, 893-896 describes characterization of shuffled subtilisins in single parameter screen: activity at 23° C./pH dependence of activity.

(2) Assays testing performance of enzymes with respect to their stability in presence of various single components. For detergents, for example, the parameter(s), in this case the components may be bleach, surfactants (anionic such as LAS, amphoteric and/or nonionic), chelants, builders, sud suppressors, polymers and others. In one embodiment of the invention a high throughput filter screening assay was developed which enables detection of residual activity upon incubation of enzyme variants for various times under various conditions. The screening could be done in a buffer including one or more important parameters or components of the detergent.

(3) Assays testing the performance of enzymes with respect to their stability when subjected to mechanical stress. This may be achieved by applying high mechanical stress to the active. Particularly, this may be done in a 96 well format ensuring high mechanical stress used for screening for resistant variants. As an example Vortex shaking of enzymes in this format leads to inactivation.

(4) Assays testing performance of enzymes with respect to their stability at different temperatures. Ness et al. (1999) Nature Biotechnology 17, 893-896 describes characterization of shuffled subtilisins in discrete dimension assay screens: thermostability measured as residual activity after 5 min incubation at 70° C. Giver et al. (1998) Proc. Natl. Acad. Sci. USA 95, 12809-12813 describes a 96-well plate thermostability screening assay used for evolution of a stabilized B. subtilis p-nitrobenzyl esterase.

(5) An important performance property of enzymes is their stain removal properties. It is useful to measure the effect on this property by one or more parameters in a discrete dimension assay. For detergent enzymes wash without presence of detergent components in screening for stain removal activities of new enzymes may be tested. Such an assay may be performed similarly to the application assay but without the parameters of the presence of other detergent components.

The actives may also be enzymes, which are to be used within other industries than the detergent industry, for example within the baking industry, the textile industry, the beverage industry, the feed industry, the food industry, the personal care industry, the paper industry, the dairy industry or any other relevant industry. Relevant discrete dimension assays to screen the enzymes in include assays testing the performance of enzymes in respect to their specific activity or stability at conditions relevant for the application of the enzyme, such as the pH, the temperature, the presence of compounds which are present during the application of the enzyme etc. Other examples include assays testing the performance of enzymes with respect to their stability or specific activity when subjected to mechanical stress or different temperature (thermostability).

In addition to the above-mentioned properties of an active relevant for the performance of the active (such as thermal and/or mechanical stability, specific activity, stain removal capability etc.), properties encompassed by the present invention also include:

(1) Facilitation of functional expression of the active in a given recombinant host for example as described in Heterologous expression of HRP in S. cerevisiae, Morawski et al. (2000), Protein Engineering 13, 377-384. Although in principle the screening system is set up similarly to screening for improved specific activities (measuring activity in culture supernatants), the specifically designed library will be significantly different. Design of a library based on codon usage, misfolding/aggregation, overcoming lacking/altered glycosylation functions/disulfide bond formation of an expression host can be done.

(2) For pharmaceutical actives, such as catalytic antibodies or antimicrobial peptides, a number of properties of the active to be measured in the discrete dimension assay may be chosen. These properties include but is not limited to facilitation of functional expression in a given recombinant host, receptor selectivity, toxicity, agonist/antagonist activity, protein binding capabilities, cell binding capabilities, in vivo half-life and plasma half-life (including stability/secretion).

(3) For actives, such as enzymes for chemical synthesis a number of properties to be measured the in discrete dimension assays of the invention may be chosen. These properties include but are not limited to resistance to organic solvents, stereo-selectivity (May et al., (2000) Nature Biotechnology 18, 317-320), substrate/product inhibition (e.g. Altamirano et al. (2000) Nature 403, 617-622.) and facilitation of enzyme immobilization.

Other properties actives may be screened for in a discrete dimension assay include altered immunogenicity and/or altered allergenicity, wherein the term “altered” is to be understood so that the immunogenicity and/or allergenicity of the active is different, i.e. either less or higher, than of a control active. For example the control may be a parent protein, and the library of proteins screened for altered immunogenicity and/or altered allergenicity may represent mutations of the parent protein.

### Collecting Nucleotide Sequences

The nucleotide sequences encoding actives with a good performance in a discrete dimension assay is isolated using standard DNA preparation techniques, or the DNA encoding the actives can be amplified directly from a clone grown as a colony on a plate or as a culture using standard PCR amplification techniques. The DNA obtained this way may subsequently be sequenced and/or directly manipulated so as to provide basis for a new library as described below.

### Mutating Nucleotide Sequences

The nucleotide sequences encoding found candidates in each of the discrete dimension assay screens are recovered and subjected to recombination/mutation to prepare a recombined/mutated gene library encoding recombined actives. The purpose of this step to combine genes encoding actives having improved properties with respect to one discrete dimension assay with genes encoding actives having improved properties with respect to another discrete dimension assay. This way actives having improved properties with respect to both discrete dimension assay may be prepared before testing the candidates in an application assay. Optionally the discrete dimension assay screening may be repeated a given number of times on the mutated actives to further reduce the number of candidate actives to be tested in the application assay. In any case, due to the initial screening of actives in the discrete dimension assays the number of candidates is considerably reduced. Recombination/mutation of the genes encoding the actives can be performed by a variety of methods:

(1) SOE-PCR as described by Kirchhoff and Desrosiers (1993) PCR Methods and Applications 2, 301-304. This method has been described supra.

(2) Preparation of synthetic gene libraries as described supra.

(3) Another method, such as Kunkel mutagenesis, employs multiple mutagenic primers to generate libraries with multiple mutated positions. This is achieved by:

(a) generating uracil containing nucleotide template encoding a polypeptide of interest

(b) synthesizing 2-50 mutagenic primers corresponding to at least one region of identity in the nucleotide template, wherein each mutagenic primer comprises at least one substitution of the template sequence (or: insertion/deletion of bases) resulting in at least one amino acid substitution (or insertion/deletion) in the amino acid sequence encoded by the starting primer,

(c) contacting the mutagenic primers with the template of (a) under conditions wherein a mutagenic primer anneals to the template sequence and extension of the primers are catalyzed by a polymerase to generate a mixture of mutagenized polynucleotides and uracil-containing templates

(d) transforming a host cell with the polynucleotide and template mixture wherein the template is degraded and the mutagenized polynucleotide replicated and thus generating a library of polynucleotide variants of the gene of interest.

(4) Still another method is DNA shuffling. DNA shuffling is the (partially) random process in which a library of chimeric genes is generated from two or more starting genes. The starting genes are heterologous such that one gene is different from any other starting gene in at least one nucleotide; i.e. the starting material can be point mutations of each other. Much more diversity can be included in the process if the parental genes differ in more positions, e.g. by representing genes encoding homologues of proteins having the same function (and structural family) but originating from different species. The latter experiment has been denoted “family shuffling” (Crameri et al, 1998, Nature, 391: 288-291).

A number of formats of carrying out this shuffling or recombination process have been described.

Examples of shuffling formats involving the random fragmentation of parental DNA followed by reassembly by PCR to new full length genes are presented in U.S. Pat. Nos. 5,605,793, 5,811,238, 5,830,721 and 6,117,679. A variant thereof in which additional diversity is added by including oligos containing mutations flanked by wt sequence in the assembly reaction has been described (spiked oligo shuffling; Crameri et al. (1996), Nature Medicine, 2,100-102).

U.S. Pat. No. 6,159,687, WO 98/41623, U.S. Pat, Nos. 6,159,688, 5,965,408 and 6,153 510 also describe methods for the in vitro recombination of genes.

WO97/07205 and WO98/28416 describe methods in which the recombination process takes place in vivo in a living cell.

Kikuchi et al (2000a, Gene 236:159-167) describe a shuffling method by which parental DNA is fragmented by restriction endonuclease digests rather than DNAse I treatment.

Kikuchi et al (2000b, Gene 243:133-137) describe a shuffling method of two parents in which complementary single stranded parental DNA of the two parents were shuffled.

Zhao et al. (1998, Nat Biotechnol 16, 258-261) describe another shuffling method (StEP; staggered extension process). In this method primers are added to denatured template genes and short fragments are produced by brief polymerase catalyzed extension. In the next cycle these fragments randomly prime the templates (template switching) and extend further. This process is repeated until full-length genes are synthesized.

A particular method of shuffling variants from the discrete dimension assays would be to follow the methods described in Crameri et al, 1998, Nature, 391: 288-291 and Ness et al. Nature Biotechnology 17: 893-896.

### Application Assays

Once a gene library encoding a reduced number of recombined/mutated actives has been prepared, the method of the invention includes testing this reduced number of candidates in an application assay, mimicking the intended real application and thus including a multiple number of interacting parameters.

The application assay should ideally be the application itself or assays designed to be as close to the application of the active as possible.

For pharmaceutical actives the application assay may be clinical tests as known to the art.

Of particular interest are enzymes for use in laundry detergents. The application assay for such enzymes should particularly represent full-scale wash conditions. Particularly the application assay is performed as described in the presently unpublished Danish patent application PA 2000 01781. This application describes methods for High Throughput washing performance tests said methods comprising:

(a) Preparing a liquid sample of less than 10 ml comprising an active,

(b) applying liquid sample to a stained surface,

(c) applying mechanical stress to the stained surface by contacting it with a body present in the liquid sample,

(d) evaluating the cleaning effect of applying solution and mechanical stress on the stained surface.

The method of this patent application was shown to be similar to real washing processes, even though it had the advantage of microtiter plates could be used for performing the test. In a particular embodiment of the present invention also the setup, devices and assemblies of PA 2000 01781 as represented though FIGS. 1 and 3-5 of that disclosure are used as an application assay.

The present invention is exemplified in the following non-limiting examples.

### EXAMPLES

### Example 1

Discrete Dimension Substrate Assay for Testing the Activity of Amylase or Protease on Filters.

Bacillus libraries were plated on a sandwich of cellulose acetate (OE 67, Schleicher & Schuell, Dassel, Germany)—and nitrocellulose filters (Protran-Ba 85, Schleicher & Schuell, Dassel, Germany) on TY agar plates with 10 micrograms per ml chloramphenicol at 37° C. for at least 21 hrs. The cellulose acetate layer was located on the TY agar plate.

Each filter sandwich was specifically marked with a needle after plating, but before incubation in order to be able to localize amylase candidates on the filter, and the nitrocellulose filter with bound variants was transferred to a container with 50% detergent base. Filters were incubated for a suitable time and temperature (e.g.16 h/37° C.). The cellulose acetate filters with colonies were stored on the TY-plates at room temperature until use. After incubation, residual activity was detected on assay plates. For detection of amylase activity, plates were containing 1% agarose, 0.2% starch in citrate buffer, pH 6.0. The assay plates with nitrocellulose filters were marked the same way as the filter sandwich and incubated for 2 hours at 37° C. After removal of the filters the assay plates were stained with 10% Lugol solution. Starch degrading amylases were detected as white spots on dark blue background and then identified on the storage plates. Identified amylases were re-screened twice under the same conditions as the first screen. For detection of residual protease activity, plates contain 1% agarose and 2% skim milk. Protease candidates yielded clearing zones.

### Example 2

Discrete Dimension Temperature Stability Assay for Actives.

The filter screening assay as outlined in example 1 was utilized as a High Throughput screen for temperature stability of AMG and alpha-amylases.

Media and Buffers and Substrate Stock Components

LBG medium (per liter: 10 g tryptone, 5 g yeast extract, 5 g NaCl, 10 g dextrose, 0.4 ml chloramphenicol)

MY25/6 medium (per liter: 25 g maltose, 10 g yeast extract, 2 g urea, 2 g MgSO4*7H2O, 2 g K2SO4, 10 g KH2PO4, 2 g anhydrous citric acid, 7 g MOPS, 0.2 g CaCl2*2H2O, 1 ml AMG trace metals, pH 7.0)

TBAB+CM plates (per liter: 10 g Bacto tryptone, 3 g Bacto beef extract, 5 g NaCl, 15 g Bacto agar, 0.1 ml chloramphenicol)

AMG trace metals (per liter: 13.9 g FeSO4*7H2O, 8.5 g MnSO4*7H2O, 14.28 g ZnSO4*7H2O, 1.63 g CuSO4, 0.24 g NiCl2*6H2O, 3 g anhydrous citric acid)

Chloramphenicol (per liter: 50 g in ethanol)

10% Skim Milk (autoclaved for 5 minutes) (per liter: 100 g non-fat dry milk powder (Lucerne brand from Safeway).

10% Calcium Chloride dihydrate (per liter: 100 g)

100 mM TRIS

10 mM Sodium Citrate

0.01% Tween-20 (per liter: 100 ml 1 M TRIS, pH 8.5, 10 ml of 1 M sodium citrate, 1 ml of 10% (w/w) Tween-20)

Hodag antifoam (as supplied),

3% Skim Milk Agar plates (per liter, 300 ml 10% skim milk, 15 grams Bacto-agar, pour 200 ml per 22 cm×22 cm QTray.

Device for Fermentation of Growth Plates Humidity controlled culture boxes are used for all microtiter plate growth conditions.

Picking Active Clones

Step 1

Appropriate libraries were plated on 3% skim milk agar plates with the following variations: (1) for high expression libraries approximately 5-8,000 colonies on Q-trays, (2) for low expression, it is necessary to supplement the agar with at least 5% LBG (or more), and to plate less (<1000). Generally overnight incubation at 37° C. is sufficient, however for low expression libraries, 2 or 3 days may be required.

Step 2

An appropriate number of 96-well plates were filled with MY25/6 plus 10 ppm Chloramphenicol at 90 microliters media per well. Active colonies were picked with a QPix colony picker by placing the tray on a white filter paper square on the light surface and setting the appropriate parameters for the colony picker. After picking, the picked colonies were grown 2-4 days in a culture box at 37° C., 350 RPM. Before screening 200 μl/well sterile 0.01% Tween-20 water was added.

Thermal Stability Screen

The discrete dimension assay consisted of treatment of a broth sample with 100 mM TRIS, pH 8.5, 10 mM sodium citrate, 0.01% Tween-20 for 10 minutes at an appropriate temperature (65° C.), followed by assays for initial and final activity. The ORCA method transferred 10 ml sample to 150 μl of treatment buffer in a 96-well polycarbonate plate. The sample was mixed and 20 μl was transferred to a flat-bottomed 96-well plate for an initial activity assay. The polycarbonate plate was transferred to a heating block (65° C.) for 10 minutes, and then the 20 μl sample was transferred to a flat-bottomed 96-well plate for a final activity assay. The substrate was 0.7% Skim Milk in 100 mM TRIS, 10 mM Calcium, 0.01% Hodag, pH 9.0 and the absorbance at 405 nm was monitored as an indicator of residual activity after heat treatment.

Recovery of Candidate Actives

After analysis of the data and determining the colonies to preserve, 100 μl of each “hit” was manually added to 1 ml of LBG in a 24-well plate and grown overnight at 37° C., 350 RPM. At this time, 250 μl of 75% glycerol was added; the plate was shaken and then frozen for future use in a recombination/mutation step. A similar procedure was used for single isolates. The exception was that a sample is streaked from a well of a 24-well plate as just described above on to TBAB/CM agar plates to grow single colonies. At least two colonies were picked into the media described above and grown/preserved as described.

### Example 3

To recombine the actives selected in each of the discrete dimension assay (such as those described in examples 1 or 2) localized random mutagenesis can be performed at each of the positions/area which on basis of the discrete dimension assays are found to be important for improved performance in said assays (these positions/areas may be identified by sequencing the nucleotide sequence of the selected actives and compare it with the sequence of a protein/peptide of a standard performance in the same assay). The overall strategy used to perform localized random mutagenesis is:

A mutagenic primer (oligonucleotide) is synthesized corresponding to the DNA sequence flanking the site of insertion, separated by the DNA base pairs defining the insertion.

Subsequently, the resulting mutagenic primer is used in a PCR reaction with a suitable opposite primer. The resulting PCR fragment is purified and extended in a second PCR-reaction, before being digested by endonucleases and cloned into the E. coli—B. subtilis shuttle vector (see below).

Alternatively, and if necessary, the resulting PCR fragment is used in a second PCR reaction as a primer with a second suitable opposite primer to allow digestion and cloning of the mutagenized region into the shuttle vector. The PCR reactions are performed under normal conditions.

Following this strategy a localized random library can be constructed of a selected active wherein every position which was found to be important for improved performance is completely randomized.

The mutations are introduced by mutagenic primers, so that all 20 amino acids are represented (N═25% of A, T, C, and G; whereas S═50% C and G. The produced PCR fragment is extended by another round of PCR by combination of an overlapping sequence with a PCR-fragment produced by PCR-amplification with primers. The extended DNA-fragments are cloned into a plasmid, and approximately ten randomly chosen E. coli colonies are sequenced to confirm the mutations designed.

The random library is transformed into E. coli by well known techniques.

The library prepared will typically contain approximately 100,000 individual clones/library.

Again approximately ten randomly chosen colonies are sequenced to confirm the mutations designed.

In order to purify a variant, the expression plasmid comprising a variant of the invention is transformed into a competent strain and is fermented according to methods known in the art typically in a medium containing 10 μg/ml Chloramphenicol (CAM).

### Example 4

Discrete Dimension Detergent Assay for Enzymes.

A library of potential detergent enzymes suitable for removing stains from textile may be screened incubating clones comprising genes encoding detergent enzymes in buffer (50 mM Glycine) at relevant pH (pH 10.4) in the presence of a pre-stained textile swatch. Enzyme candidates may be identified as those removing stain components when measuring the light reflectance of the stained swatch after treatment with the enzymes.

### Example 5

High Throughput Screening Application Assay for Detergent Alpha-amylases with Better Wash Performance.

One industrial application of alpha-amylases is removal of starch based soils from laundry.

Preparation of Textile Swatches Coated with Starch

Unmodified starch from a natural source was mixed with small amounts of fluorescently labelled starch and coated on a solid phase. The natural starch source can be flour derived from e.g. potato, corn, or rice. Especially rice flour has been observed to provide a good correlation to higher scale wash trials. The solid phase may be twill, as twill has been found to provide good correlation to larger scale washes and has good handling properties. The overall starch concentration as well as the ratio between labelled and unlabelled starch can be varied over a wide range, but we found that 5% (w/v) unmodified starch and 0.025% (w/v) fluorescin 5-isothiocyanate (FITC) labelled potato starch (50-300 glucose units per FITC molecule) to provide the best compromise between sensitivity and response level (FITC was obtained from Sigma).

The starches was suspended in water and boiled for 10 min, alternatively jet-cooked for 5 min at 105° C. and 2 bar. After cooling, a selected textile was soaked with the cooked suspension, excess slurry removed by rolling, and the textile was dried either overnight at ambient temperature or for a few minutes at high temperature, e.g. 70° C., at high air velocity, e.g. 12 m/s. We found that automation of this coating procedure significantly reduced coating variation. By use of a coating machine to continuously coat several hundred meters at a time, subsequently performed assays with repeated dose-response curves exhibited considerably less variation compared to non-continuous coatings.

Growth of Bacillus Cells Secreting Alpha-amylase Variants

Bacillus cells secreting alpha-amylase variants were grown in 2xYT and 6 μg/ml Chloramphenicol for three days at 37° C. and 240 rpm, in a culture box (see example 2).

High Throughput Screen for Detergent Alpha-amylases with Better Wash Performance in Application Assay

Dry starch coated textile was punched into wells of a polystyrene microtiter plate, preferentially of the 96-well format. Assays were performed by first applying a detergent solution, e.g. 150 μl, to each well. A range of detergents could be used such as liquid detergent or powder detergent dissolved in water. Water hardness was controlled by the addition of a desired amount of calcium and magnesium ions. The assay itself was insensitive to water hardness over a wide range, e.g. 0-30° dH. If a detergent that contained enzymes was used, these enzymes could be inactivated by e.g. heating the detergent to 85° C. for 5 min prior to the assay. Furthermore, the detergent could be centrifuged and/or filtered before use to minimize particulate matter. However, no assay interference due to unremoved particles was observed with detergents used in the range of dosages recommended by the manufacturers.

The intrinsic pH of detergents was controlled with added buffering capacity by adding e.g. glycine or 3-[cyclohexylamino]-1-propane-sulfonic acid (CAPS). This was an important quality enhancing measure, since the high throughput screen used culture supernatants as the source of expressed enzyme. Growth media normally contained buffer components to ensure the optimal growth pH, which were rarely equal to the high pH (often pH 9-11) present in dissolved detergents.

Culture medium from above was added immediately after dispensing the detergent. It was preferred to use a small amount of enzyme solution compared to detergent/buffer, in order to minimize artifacts caused by e.g. a rich growth medium. For example, we routinely applied 15 μl of enzyme solution to 150-μl detergent.

Following addition of detergent and enzyme (culture medium), the simulated “wash” took place. Application of mechanic motion such as vigorous shaking (e.g. 240 rpm) at this point strongly promoted the effect of tested enzymes, thus creating a good dynamic range for ranking candidate enzymes. Incubation times from 5 min to 2 h were tested at this step. When screening for amylases at low-temperature conditions, we have found 15 min “washing” to provide a good window of difference between “good” or “bad” candidates.

Heating could be applied during incubation to simulate the heating of water during machine washing used in many parts of the world. For example, simulation of European washing conditions could include heating up to 40 or 60° C. This heating could be introduced gradually by for example placing the ambient temperature microtiter tray in a shaking incubator set to the appropriate temperature. By this approach, a heat-up profile was generated. Alternatively, if constant high temperature was desired, heated buffers could be added or insulated tubings and thermostated surroundings could be employed.

The assay responses could be read by transferring the wash solution to another microtiter plate and measure released fluorescence in this solution. Alternatively, the wash solution could be completely removed and the response measured as residual fluorescence of the swatch.

By using the conditions described above, the two approaches generated almost exact mirror image data, meaning that a high degree of released fluorescence in a well is reflected by a low degree of residual fluorescence on the corresponding swatch and vice versa. We preferred the last method, as this only requires one well per assay, thus ensuring minimal amounts of operations leading to maximal throughput in e.g. a robotic set-up.

The growth conditions used ensured a reasonably uniform growth of identical clones in different wells of the plate. However, large differences in expression level between different variants still could be observed. We have found that amylase variants with different performance in the final application released different amounts of fluorescent stain from the swatch in the wash performance assay, even when the enzyme was used in saturating concentrations. Therefore, the screen was set up using very high concentrations of enzyme. Under these conditions the stain removal efficiency depended only minimally on the enzyme concentration within a broad range; consequently, differences in expression levels between variants had little effect on the performance of the individual variant in the high throughput screening application assay.

When applying the assay to libraries of amylase producing clones, “hits” were defined as wells of the plate wherein the measurements exceeded a given assay response relative to a selected benchmark amylase.

Results

In one application assay orange colored rice starch textile was shaken vigorously in 60 mL Asia-Pacific model detergent containing an amylase variant, and the reflectance measured. Table 1 below shows the correlation between the performance of three different amylase candidates (1, 2, and 3) in the swatch assay (the High Throughput Screening set-up) and the conventional mini-wash test. Application dosage of 0.2 μg/ml amylase was used for the mini wash, while approx. 2 μg/ml final dosage was used in the swatch assay (the average concentration from culture supernatants in the High Throughput Assay).

The improvement factor in a given assay is given by the performance of the variant amylase divided by the performance of a given benchmark amylase. Obviously, the improved amylase (amylase 3) performed better in both the swatch assay and the mini-wash.

| | Amylase 1 | Amylase 2 | Amylase 3 |
|---|---|---|---|
| Swatch assay | 1.0 | 0.4 | 1.1 |
| Mini-wash | 1.0 | 0.3 | 1.3 |

1. A screening method for identifying actives with improved performance comprising:
(1) screening a library of actives encoded by nucleotide sequences in at least two different discrete dimension assays employing selected parameters and selecting actives exhibiting improved properties in the discrete dimension assays,
(2) collecting nucleotide sequences encoding the selected actives,
(3) mutating collected nucleotide sequences,
(4) screening actives encoded by the mutated nucleotide sequences in an application assay and selecting actives, which exhibit improved performance.

2. The method of claim 1, wherein the active is a peptide or a protein.

3. The method of claim 2, wherein the active is selected from the group consisting of bio-catalysts, preferably enzymes, therapeutic agents, preferably hormones, insulin, growth factors, coagulation factors, antibodies, receptors or antibiotics and biocides, preferably herbicides, pesticides and fungicides.

4. The method of claim 1, wherein the library is prepared by preparing a gene library comprising nucleotide sequences encoding actives and expressing the gene library in an expression system.

5. The method of claim 4, wherein the expression system is a cellular or an in vitro expression system.

6. The method of claim 4, wherein the gene library has been subjected to manipulation selected from the group consisting of Error prone PCR, SOE-PCR, Oligonucleotide directed randomisation, Combinatorial site-directed mutagenesis, synthetic gene libraries, Kunkel mutagenesis, preparation and gene shuffling.

7. The method of claim 1, wherein the selected parameters are selected from the group consisting of temperature, pH, Ca2+, bleach, surfactants, LAS, chelants, builders, sud suppressors, polymers and mechanical stress or a combination thereof.

8. The method of claim 1, wherein the property of the active is selected from the group consisting of specific activity, physiochemical stability, stain removal capabilities, facilitation of functional expression in a recombinant host, receptor selectivity, toxicity, agonist/antagonist activity, protein binding capability, cell binding capability, plasma half-life, in vivo half life in animals including man, altered immunogenicity, altered allergenicity, resistance to organic solvents, stereoselectivity, substrate or product inhibition and facilitation of enzyme immobilization.

9. The method of claim 1, wherein the method for mutation of collected nucleotide sequences is selected from the group consisting of SOE-PCR, preparation of synthetic gene libraries, Kunkel mutagenesis and gene shuffling.

10. The method of claim 1, wherein the application assay is selected from the group consisting of a washing process and clinical tests.

11. A method according to any of the preceding claims, wherein the active is isolated from agar plates containing discrete arrays of holes producing clearing zones or other visible zones identifying the variant as an active variant.

12. A method according to any of the preceding claims, wherein at least two different libraries of actives in step (1) are screened in a discrete dimension assay.

13. An active obtained by the screening method of claim 1.
